(19) United States
(12) Patent Application Publication
(10) Pub. No.: US 2015/0238129 A1
(43) Pub. Date: Aug. 27, 2015

(54) Method for Producing a Sensor Instrument, and Sensor Instrument

(71) Applicant: SIEMENS AKTIENGESELLSCHAFT, München (DE)

(72) Inventors: Stefan Förtsch, Erlangen (DE); Rainer Kuth, Hochstadt (DE)

(21) Appl. No.: 14/423,129

(22) PCT Filed: Aug. 19, 2013
(86) PCT No.: PCT/EP2013/067242

§ 371 Date: Feb. 21, 2015

(30) Foreign Application Priority Data
Aug. 21, 2012 (DE) 102012214801.9

(51) Int. Cl. A61B 5/1473; B29C 70/88; B29C 70/68; A61B 5/00; B29C 70/74

(52) U.S. Cl. CPC A61B 5/1473; A61B 5/4233; A61B 5/4238; A61B 5/4255; B29C 70/74; B29C 70/683; B29C 70/885; A61B 2562/12; B29L 2009/008

(57) ABSTRACT

In a method for producing a sensor instrument (2) having an ammonia-sensitive sensor (8), said sensor being designed to examine the mucous membrane of the esophagus, stomach, and duodenum of a patient for infection with bacteria, at least one electrically conductive wire (10) is embedded in a non-conductive matrix (12) and then an end of the wire (10) is exposed by way of grinding.

The invention relates to a method for producing a sensor instrument and to a corresponding sensor instrument.

A possible reason for discomfort of a patient in the region of the upper gastrointestinal tract is an infection with Helicobacter pylori bacteria.

DE 10 2010 006 969 A1, which can be traced back to the applicant, has disclosed a test method, with the aid of which a patient can be examined for such an infection. To this end, use is made of a gastroscope with an insertion tube, at the distal end of which a sensor, which reacts sensitively to ammonia, is arranged. Here, use is made of the fact that Helicobacter pylori bacteria split urea into carbon dioxide and ammonia by means of the urease enzyme and that ammonia is typically only detectable in relevant amounts in the stomach of a patient in the case of an infection with Helicobacter pylori bacteria. Therefore, the presence of an increased amount of ammonia and, as a consequence, an infection with Helicobacter pylori bacteria can be deduced in the case of a corresponding reaction of the sensor, which is positioned in the stomach of the patient.

The basic functional principle of the sensor was presented, inter alia, within the scope of the presentation “Immediate detection of Helicobacter infection with a novel electrochemical system” (Gastroenterology, volume 138, issue 5, supplement 1, pages S-114, May 2010) by Helmut Neumann, Stefan Foertsch, Michael Vieth, Jonas Mudter, Rainer Kuth and Markus F. Neurath during the “DIGESTIVE DISEASE WEEK 2010”. According thereto, a change in an electric variable is registered metrologically when an electrode pair comes into contact with ammonia, wherein one electrode of the electrode pair reacts chemically with the ammonia.

A gastroscope is a special endoscope for examining the mucous membrane of the esophagus, stomach and duodenum and it is therefore a relatively complex medical instrument, which is produced with a relatively high technical and financial outlay. Particularly as a result of continuously increasing costs in the health sector as well, it is advantageous to configure sensor instruments for the medical field in such a way that the production thereof can be carried out as easily and cost-effectively as possible.

Proceeding from this, the invention is based on the object of specifying a simple method for producing a sensor instrument and a corresponding sensor instrument.

According to the invention, this object is achieved by a method comprising the features of claim 1. The dependent claims contain partly advantageous and partly independently inventive developments of this invention. According to the invention, this object is moreover achieved by a sensor instrument comprising the features of claim 10.

The method serves for producing a sensor instrument, wherein, for the purposes of manufacturing an ammonia-sensitive sensor, at least one electrically conductive wire or electrically conducting lead is embedded in a nonconductive matrix and, subsequently, one end of the wire is exposed by grinding. Here, the ammonia-sensitive sensor is embodied for examining the mucous membrane of the esophagus, stomach and duodenum of a human or else animal patient to see whether it is infected with bacteria which, due to a corresponding metabolic reaction, emit ammonia to the surroundings thereof, i.e., also, bacteria of the genus Helicobacter, such as e.g. Helicobacter pylori, Helicobacter heilmannii or “Candidatus Helicobacter suis”. This procedure was found to be particularly expedient for the manufacturing since, using it, the requirements on the sensor instrument can be satisfied without major technical outlay.

Here, the wire or the lead is preferably embedded in the nonconductive matrix in such a way that the nonconductive matrix securely surrounds the wire such that, when the free end is immersed into a liquid, said liquid cannot enter between the matrix and wire.

At the end of the production method, the ammonia-sensitive sensor comprises at least two wires or leads, respectively embedded in a nonconductive matrix, wherein the two embedded wires are e.g. adhered to one another for predetermining a fixed spacing. Alternatively, two electrically conductive wires or leads are embedded in the nonconductive matrix with a predetermined spacing and, subsequently, respectively one end of the wires is exposed by grinding. Thus, instead of individually embedding the wires in the matrix and then fastening these to one another in a second manufacturing step in order to form the sensor, the wires are, in this case, embedded together in the matrix in one manufacturing step with a predetermined spacing.

In accordance with one advantageous method variant, at least one of the wires is ground flat, perpendicular to the lead central axis of the wire, at one end and both wires are preferably processed further in this manner. A surface, typically a circle-shaped end face, is predetermined by this grinding flat, which has an expedient effect on the subsequent method steps when producing the sensor instrument and on the function of the sensor.

Furthermore, the sensor instrument and, in particular, the sensor are preferably deburred. What is important here, inter alia, is to ensure that the sensor instrument, which is provided for insertion into the gastrointestinal tract of a patient, includes no sharp edges, corner etc., by means of which a patient could be injured. The wires used for the sensor preferably consist of a comparatively simple and cost-effective stainless steel or copper alloy and are expediently coated, in particular electroplated, during a method step. Here, the coating is preferably only undertaken at the exposed ends. As a result of coating, the use of high quality and expensive materials is restricted to a minimum. Here, for each sensor, one wire is coated with silver for forming an electrode and one wire is coated with gold or platinum for forming a reference electrode. If the two wires were embedded together in the nonconductive matrix, the wire which is not intended to be coated within the scope of the coating process is initially coated with a protective lacquer for the purposes of avoiding an unwanted coating. As a result of this, the two wires can be coated differently, even if they can only be immersed together in an electrolytic bath.

Depending on the measurement method that is ultimately to be realized, at least the electrode, i.e. the wire coated with silver, is furthermore immersed in HCl for passivation purposes such that a silver chloride layer, which chemically reacts in the case of contact with ammonia, forms on the surface.

In order to avoid an unwanted reaction, at least part of the sensor, i.e., in particular, the electrode, is coated with a protective layer or protective lacquer which is water soluble or stomach-acid soluble. As a result, the sensor, and hence the sensor instrument, can be stored over a relatively long period of time without further special protective measures. Then, no reduction in the effectiveness of the sensor instrument caused by aging is to be expected. A corresponding protective lacquer is preferably produced from NaCl or NaHCO₃.

What must moreover be considered during the production of the sensor instrument is that the materials used herein have to be biocompatible and that, even in the case of contact with stomach acid, no substances that could cause intolerance or even symptoms of poisoning in the patient may dissolve out either. It is for this reason too that e.g. polymethylmethacrylate, polyoxymethylene, polycarbonate or a thermoset polymer made of an epoxy resin and a hardener is used for the nonconductive matrix.

A sensor instrument manufactured with the aid of the method described here has an ammonia-sensitive sensor at the end of the manufacturing process, which sensor is embodied for examining the mucous membrane of the esophagus, stomach and duodenum of a patient for infection with bacteria, wherein the sensor comprises two electrically conductive wires embedded in a nonconductive matrix, the ends of which wires are exposed and are either manufactured from different materials or coated differently. The ends serve firstly as electrode and secondly as counter electrode and an electrochemical processes is started by immersing the sensor into the stomach content of a patient, which contains stomach acid acting as an electrolyte, depending on whether or not, moreover, a relevant amount of ammonia is present such that a change in an electrical variable is measurable with the aid of the sensor and a suitable evaluation unit.

Preferably, there is a measurement of the electrical resistance of the sensor in this case, wherein a high resistance is initially present due to the silver chloride layer. If a patient now has an infection, e.g. with Helicobacter pylori bacteria, there is an increased concentration of ammonia, caused by the bacteria, in the stomach content of the patient, which ammonia reacts with the water-insoluble silver chloride. In the process, a water-soluble silver diamine complex is produced, as a result of which the silver chloride layer is removed from the electrode. As a result of this, the silver layer lying therebelow is exposed and the electrical resistance of the sensor reduces. Exemplary embodiments of the invention will be explained in more detail below on the basis of a schematic drawing. In detail:

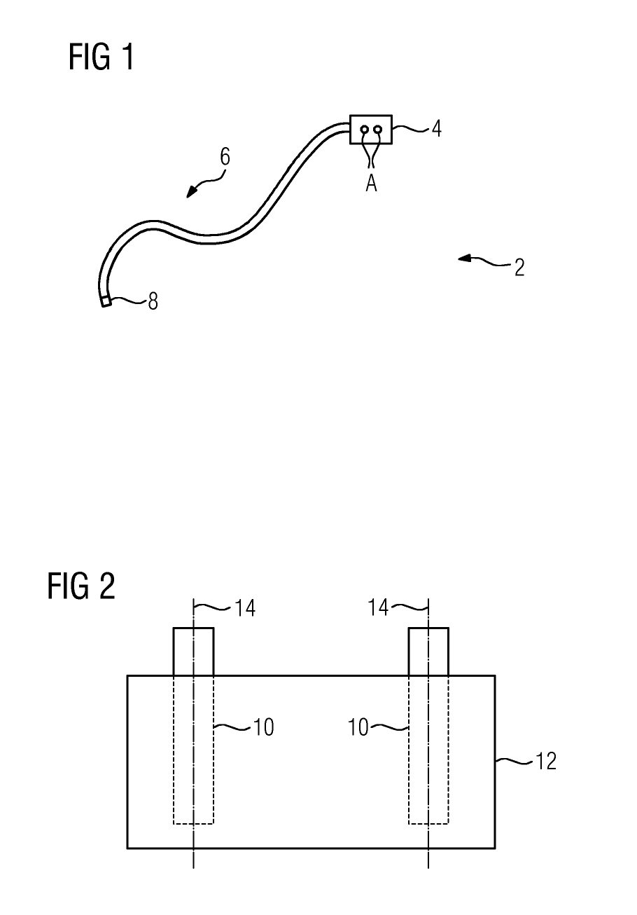

FIG. 1 shows a side view of a sensor instrument comprising a sensor,

FIG. 2 shows a magnified view of the unfinished sensor after embedding two wires in a matrix,

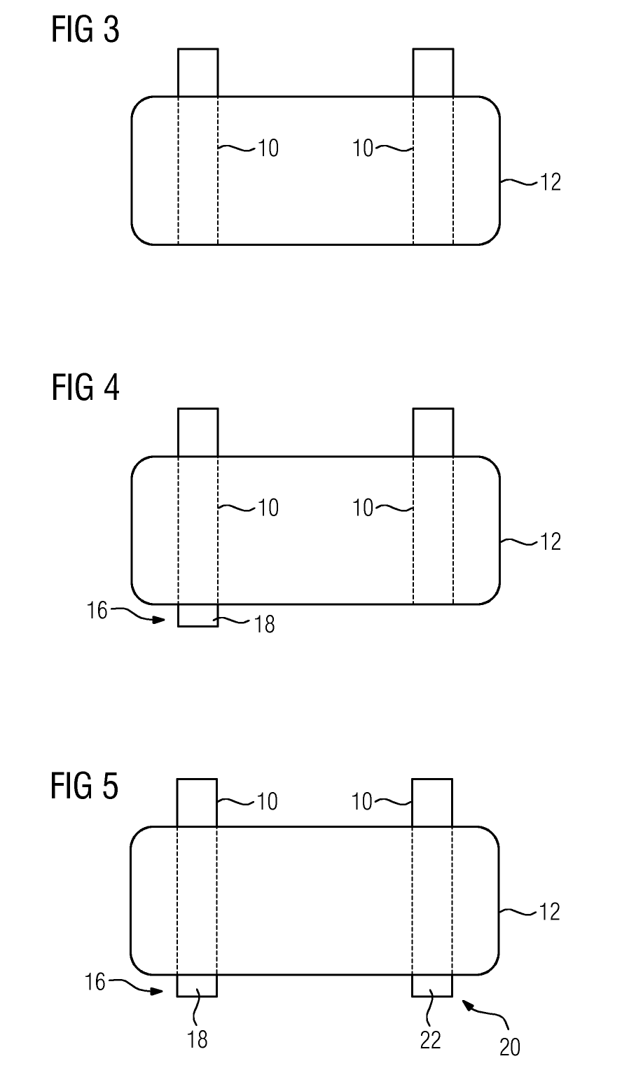

FIG. 3 shows a magnified view of the unfinished sensor after exposing one wire end in each case,

FIG. 4 shows a magnified view of the unfinished sensor after coating one of the ends of the wires with silver,

FIG. 5 shows a magnified view of the unfinished sensor after coating the other end with gold,

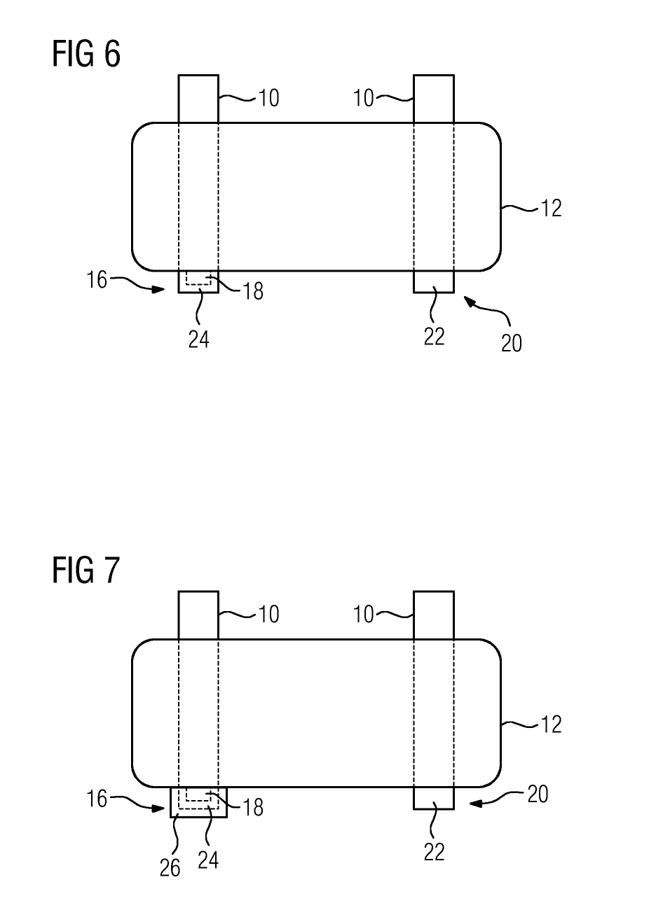

FIG. 6 shows a magnified view of the finished sensor after passivation of one of the ends of the wires, and

FIG. 7 shows a magnified view of the finished sensor after coating one of the ends of the wires with a protective lacquer.

Parts corresponding to one another have respectively been provided with the same reference sign in all figures.

The method described below serves, in an exemplary manner, for producing a sensor instrument 2 as depicted in FIG. 1. The latter is constructed from an evaluation unit 4 comprising an optical display A and a catheter probe 6 comprising an ammonia-sensitive sensor 8.

The end of the catheter probe 6 lying opposite the evaluation unit 4 and acting as a sensor 8 is depicted in a magnified manner in FIGS. 2 to 7, wherein the individual images show the state of the catheter probe 6 after the various manufacturing process steps.

In order to produce the catheter probe 6, two stainless steel wires 10 are initially embedded in a matrix 12 made of polycarbonate—as indicated in FIG. 2—in such a way that, firstly, the spacing between the stainless steel wires 10 is predetermined and that, secondly, the matrix 12 lies securely against the stainless steel wires 10. This prevents a liquid, into which the catheter probe 6 is immersed, from being able to enter the sensor 8 and propagate between the matrix 12 and the stainless steel wires 10. By way of example, the two leads of a two-core electrical cable with insulation are used as stainless steel wires 10, wherein the insulation has been removed from one end of the cable for embedding purposes.

In a subsequent method step, the ends of the two stainless steel wires 10 are exposed by a grinding process and ground flat perpendicular to the central axis 14 of the stainless steel wires 10, and the catheter probe 6 is deburred, particularly on the end side. What is important here, inter alia, is to ensure that the catheter probe 6, which is provided for insertion into the gastrointestinal tract of a patient, includes no sharp edges, corner etc., by means of which a patient could be injured. As an alternative to the illustration in accordance with FIG. 3, the surface of the wires 10, which are ground flat, terminates flush with the matrix 12.

In a further sub-process, one of the stainless steel wires 10 is provided with a silver layer 18 for forming an electrode 16. Here, the coating is undertaken with the aid of an electroplating method, during which the second stainless steel wire 10, which is not to be coated in this sub-process, is coated by a protective lacquer. After electroplating and the removal of the protective lacquer, the catheter probe 6 is in the state as indicated in FIG. 4. A production step is provided in a complementary manner, during which, in a manner analogous to the preceding process step, the second stainless steel wire 10 is provided with a gold layer 22 for forming a reference electrode 20, wherein, once again, the electrode 16 not to be coated within the scope of this process step is provided with a protective lacquer.

After the electroplating of the two stainless steel wires 10 in order to form, firstly, the electrode 16 and, secondly, the reference electrode 20, there is a passivation of the electrode 16 by immersion in a hydrochloric acid bath, as a result of which a silver chloride layer 24 forms on the surface of the electrode 16. This situation is depicted in FIG. 6.

Finally, the electrode 16 is coated by sodium chloride protective lacquer 26, at least in the region of the silver chloride layer 24, in order to avoid unwanted aging effects during the storage of the sensor instrument 2.

The invention is not restricted to the exemplary embodiment described above. Rather, a person skilled in the art can also derive other variants of the invention therefrom, without departing from the subject matter of the invention. Furthermore, in particular, all individual features described in conjunction with the exemplary embodiment are also combinable with one another in a different manner, without departing from the subject matter of the invention.

1. A method for producing a sensor instrument (2), wherein, for the purposes of manufacturing an ammonia-sensitive sensor (8), which is embodied for examining the mucous membrane of the esophagus, stomach and duodenum of a patient for infection with bacteria, at least one electrically conductive wire (10) is embedded in a nonconductive matrix (12) and, subsequently, one end of the wire (10) is exposed by grinding.

2. The method as claimed in claim 1, wherein exactly two electrically conductive wires (10) are embedded into the matrix (12) at a predetermined distance and wherein, subsequently, respectively one end of the wires (10) is exposed by grinding.

3. The method as claimed in claim 1 or 2, wherein at least one wire (10) is ground flat at one end.

4. The method as claimed in one of claims 1 to 3, wherein deburring of the sensor (8) is undertaken.

5. The method as claimed in one of claims 2 to 4, wherein one wire (10) is coated, in particular by electroplating, with silver for forming an electrode (16) and one wire (10) is coated, in particular by electroplating, by gold or platinum for forming a reference electrode (20).

6. The method as claimed in claim 5, wherein the electrode (16) is immersed in hydrochloric acid for passivation purposes.

7. The method as claimed in one of claims 1 to 5, wherein the sensor (8) is at least partly coated by protective lacquer (26), which is soluble to water or stomach acid.

8. The method as claimed in claim 7, wherein the protective lacquer (26) is produced from NaCl or NaHCO₃.

9. The method as claimed in one of claims 1 to 5, wherein polymethylmethacrylate, polyoxymethylene, polycarbonate or a thermoset polymer made of an epoxy resin and a hardener is used for the matrix (12).

10. The sensor instrument (2) comprising an ammonia-sensitive sensor (8), which is embodied for examining the mucous membrane of the esophagus, stomach and duodenum of a patient for infection with bacteria, wherein the sensor (8) comprises two electrically conductive wires (10) embedded in a nonconductive matrix (12), the ends of which wires are exposed.
